(19) United States
(12) Patent Application Publication
(10) Pub. No.: US 2008/0283059 A1
(43) Pub. Date: Nov. 20, 2008

(54) DEVICE AND PROCESS FOR RESPIRATING A PATIENT

(75) Inventors: Erich Siegel, Bad Schwartau (DE); Wilfried Buschke, Lubeck (DE)

Correspondence Address: MCGLEW & TUTTLE, PC, P.O. BOX 9227, SCARBOROUGH STATION, SCARBOROUGH, NY, 10510-9227, US

(73) Assignee: Drager Medical AG & Co. KG, Lubeck (DE)

(21) Appl. No.: 12/121,964

(22) Filed: May 16, 2008

(30) Foreign Application Priority Data
May 18, 2007 (DE) 10 2007 023 246.4

(51) Int. Cl. A61M 16/12; A61M 16/01

(52) U.S. Cl. 128/203.25; 128/204.18

(57) ABSTRACT

For the first time ever, a device (1) for shortening the wake-up phase of an anesthetized patient (Pa) is provided. Furthermore, a process is provided for operating a device (1) as well as a process is provided for respirating a patient (Pa) with gaseous anesthetic (17).

### CROSS REFERENCE TO RELATED APPLICATIONS

This application claims the benefit of priority under 35 U.S.C. §119 of German Patent Application DE 10 2007 023 246.4 filed May 18, 2007, the entire contents of which are incorporated herein by reference.

### FIELD OF THE INVENTION

The present invention pertains to a device for respirating a patient with gaseous anesthetic. The present invention pertains, furthermore, to a process for operating a device for respirating a patient with gaseous anesthetic, as well as to a system for respirating a patient with gaseous anesthetic.

### BACKGROUND OF THE INVENTION

The waking up of a patient after anesthesia requires that the patient's respiratory center be stimulated. This stimulation is carried out, as a rule, by intentionally raising the carbon dioxide (CO₂) level in the breathing gas. However, such an increase is, in principle, in conflict with the reduced oxygen (O₂) supply of the patient, which is associated herewith, and also with the necessary wash-out of the volatile anesthetic or anesthetics at the end of the anesthesia.

The waking-up process from an anesthesia is usually controlled such that after the anesthetic agent is turned off or its concentration is reduced, respiration is switched over to manual respiration. It is achieved by a slight hypoventilation that the CO₂ concentration in the breathing gas rises and the respiratory center is stimulated hereby. One difficulty in this procedure is that the hypoventilation slows down the likewise necessary wash-out of the volatile anesthetic, so that the waking-up process cannot be carried out in a time-optimal manner.

Furthermore, there are different, relevant wash-out times and such wash-out times are to be taken into account, especially in case of using more than one anesthetic, depending on the type, combination and duration of administration of the drugs used. It is hardly possible so far to predict the time to wake up precisely in the case of complex dosing schemes.

### SUMMARY OF THE INVENTION

The object of the present invention is therefore to provide a device and a process for shortening the phase of waking up of a patient from anesthesia. Another object of the present invention is to offer clues for the depth of anesthesia and/or the foreseeable time to wake up the patient at the end of the anesthesia.

The object according to the present invention is accomplished by the combination of the features of the device according to the present invention for respirating a patient with gaseous anesthetic. The device has at least one hypoventilation means for bringing about hypoventilation of the patient and at least one means for checking the spontaneous breathing of the patient once hypoventilation has been brought about. This is achieved by means of the at least one hypoventilation means of bringing about hypoventilation that the CO₂ concentration in the lungs of the patient rises and the respiratory center is stimulated hereby. The present invention covers any means of bringing about an increase in the CO₂ concentration, i.e., hypoventilation, which is known to the person skilled in the art. Such an increase in the CO₂ concentration may be brought about, for example, by reducing the respiration rate. However, an increase in the CO₂ content in the breathing gas may also be brought about by deactivating a CO₂ absorber possibly provided in the respiration system.

After thus bringing about hypoventilation of the patient, the spontaneous breathing of the patient is checked by means of at least one checking means for checking the spontaneous breathing to determine whether the patient is already sufficiently prepared for waking up. Criteria for such a state close to waking up can be set variably and individually depending on the particular patient and have one or more of the following conditions a) through f). Thus, the criterion of sufficient spontaneous breathing and/or of a sufficiently low end-expiratory gaseous anesthetic concentration can be met if:

a) the end-tidal CO₂ value has returned to a normal value;

b) uniform breaths are recorded;

c) the spontaneous minute volume is more than 60% of the last mandatory respiration setting;

d) the end-tidal anesthetic concentration has dropped to less than 30% of the mean value measured during the anesthesia;

e) the oxygen saturation in the blood is more than 97%; and/or

f) the bispectral index in the electroencephalogram is greater than 60, should such a measured value be determined in the particular case.

It is noted that the criterion may comprise one or more of the above-mentioned individual criteria a) through f). Furthermore, it is noted that the values given above for the spontaneous minute volume, the oxygen saturation, the anesthetic concentration and the like can be made dependent on other values or on values of another level.

If the criterion of a sufficient spontaneous breathing and/or of a sufficiently low end-expiratory anesthetic gas concentration is met, a fresh gas flow can be set at, e.g., 6 L/minute or higher, and the respiration can pass over into a spontaneous breathing mode. Furthermore, an instruction can be sent to the user or the attending physician that the wake-up process has ended.

By means of the device according to the present invention and its means for bringing about hypoventilation and for checking spontaneous breathing during hypoventilation, the checking can be carried out as often as desired and repeated at time intervals that can be set as short as desired until a transition into the spontaneous breathing mode has taken place.

The present invention is advantageously characterized in that the physician or user in charge of the device according to the present invention now has the possibility of predicting the transition to spontaneous breathing with a hitherto unknown accuracy and therefore unknown safety for the patient. The present invention leads, furthermore, to a shortening of the wake-up phase and hence to a termination of anesthesia that causes less discomfort for the patient. Furthermore, the present invention advantageously relieves the staff and leads, moreover, to a shortening of the entire surgical process, for which the anesthesia was carried out, and hence to a shorter occupation of the operating room, as well as to an overall reduction of the duration of anesthesia and to an anesthesia that is therefore felt by the patient to be more pleasant. Due to the fact that the point in time at which the transition to spontaneous breathing takes place can be determined more accurately according to the present invention, it is, furthermore, possible to prematurely withdraw the anesthetics used, and anesthetics can therefore also be saved according to the present invention. Not only does such a saving of drugs for maintaining the anesthesia make anesthesia less expensive, but, as has been shown by experience, also causes less discomfort for the patient, which implies further advantages of the present invention.

Thus, in an especially preferred embodiment, the device according to the present invention has a carbon dioxide absorber or CO₂ absorber and at least one switchable bypass position for the CO₂ absorber. By switching the carbon dioxide absorber into its bypass position, hypoventilation of the patient is brought about, as it was defined and discussed above.

The use of a CO₂ absorber together with a bypass position advantageously permits the CO₂ in the breathing gas of the patient to be increased even in case of respirators that have a CO₂ absorber for lowering the CO₂ level in the rebreathed breathing gas during a middle section of anesthesia as a permanently integrated CO₂ absorber.

Another preferred embodiment according to the present invention has at least one detector for the above-mentioned bypass position. This detector advantageously ensures that the device, which may have a complicated alarm system for avoiding excessively high and excessively low concentrations of the anesthetics but also of the CO₂ content in the breathing gas, will not generate an unintended alarm at least when the device intentionally provokes an increase in the CO₂ level in the breathing gas to bring about hypoventilation of the patient.

In another preferred embodiment, the device according to the present invention has a combination cartridge for the carbon dioxide absorber or CO₂ absorber and for a gaseous anesthetic absorber. Both CO₂ absorption during the ongoing anesthesia during rebreathing by the patient and, in a bypass position of the CO₂ absorber by switching over the combination cartridge to the anesthesia absorber, wash-out of the anesthetics can take place especially advantageously by means of this combination cartridge in a compact manner in a device for respirating a patient with gaseous anesthetic. Consequently, while the CO₂ absorber is in the bypass position, in which it is not connected into the respiration circuit, wash-out of the anesthetic or anesthetics used thus takes place by means of the gaseous anesthetic absorber connected into the respiration circuit instead of the CO₂ absorber. Thus, the same space available for installation can be used both to receive the CO₂ absorber and to receive the activated carbon filter without these mutually interfering with one another functionally. A device according to the present invention thus designed is therefore of a welcome compact nature.

The combination cartridge may be equipped such that the CO₂ absorber and the activated carbon filter or absorber for the anesthetics are connected simultaneously into the breathing gas flow. However, at least the CO₂ absorber or the gaseous anesthetic absorber has a reduced activity only in such a case. Thus, wash-out of the anesthetics and an increase in the CO₂ concentration can take place simultaneously.

It is clear to the person skilled in the art that the invention is also directed towards the combination cartridge and its use alone, i.e., regardless of other features, the combination cartridge alone is an aspect of the invention.

The combination cartridge may have a manual changeover switch between the CO₂ absorber and the anesthetic absorber. However, it is also covered by the present invention in the design in which it can be changed over automatically. Another preferred embodiment according to the present invention has a detector for the position of the switch of the combination cartridge. It is indicated to the device by means of this detector whether the combination cartridge is in the position in which the gaseous anesthetic absorber is connected into the breathing gas circuit or in a position in which the CO₂ absorber is connected into the breathing gas circuit, or whether the combination cartridge is in an intermediate position. As was already discussed above, alarm reports can be prevented by means of this detector, because the information on the current position of the combination cartridge is available in the device of this embodiment according to the present invention. The cause of certain values or monitoring results, which would otherwise lead to an alarm, is thus known.

In yet another preferred embodiment according to the present invention, the device has a preparatory process means for carrying out a preparatory process. Waking up of the patient from an anesthesia is prepared by this preparatory process means, and the composition of the anesthetics administered can be optimized by the preparatory process means such that early waking up is possible and/or a sufficient wash-out of every anesthetic used (or at least of one of the anesthetics) takes place within the narrowest possible time window. Such a preparatory process means can make possible the controlled supply of at least one first anesthetic and of a second anesthetic in such a way that the quantity of these anesthetics can be set. Furthermore, the preparatory process means may have a data processing unit with a display means belonging to the data processing unit, which is set up to perform a pharmacokinetic model calculation and an action module for storing the course of at least one anesthesia action parameter as a function of the concentrations of the anesthetic or anesthetics.

The preparatory process means may contain, furthermore, a display module for displaying an action diagram, in which the concentration of the anesthetics as well as the sequence of the concentration data of the past course of anesthesia are displayed. Such a means is described in the German Patent DE 10 2004 050 717 B3 (corresponding to U.S. patent application Ser. No. 11/250,026). Reference is expressly made hereby to the overall disclosure of German Patent DE 10 2004 050 717 B3 (corresponding to U.S. patent application Ser. No. 11/250,026, which is hereby incorporated by reference). The subject of the patent specification mentioned above may be provided and used in connection with the device according to the present invention in any desired embodiment. The wash-out of the anesthetics can be optimized by means of this embodiment according to the present invention already before the start of the wake-up operation to the shortest possible wake-up time by increasing the relative or absolute concentration of the short-acting anesthetics and lowering the relative or absolute concentrations of the long-acting anesthetics.

In another preferred embodiment, a device according to the present invention for respirating a patient may be designed such that the wake-up process is started by only pressing a button and the anesthetic dispensing systems stop the dispensing, the bypass function is turned on manually or automatically and the wake-up algorithm is started.

Another preferred embodiment has a combination cartridge. The combination cartridge may comprise a container with two separate components. The container may be designed such that the CO₂ absorbent and the activated carbon can be refilled in a first embodiment. This embodiment is therefore a reusable combination cartridge. In another embodiment, the combination cartridge may be filled up with a defined quantity of CO₂ absorbent and/or activated carbon as a representative of a gaseous anesthetic absorber. This cartridge must be replaced after depletion. Provisions are made in a preferred embodiment of the combination cartridge for providing suitable membranes as filters for the CO₂ absorption and/or gaseous anesthetic absorption.

An alternative embodiment is provided with an intravenously administered anesthetic, e.g., propofol. Activation of the activated carbon filter or of the gaseous anesthetic absorber is not necessary in this embodiment, but a gaseous anesthetic absorber, which is used as a bypass of the CO₂ absorber, may be present here as well. The presence of a gaseous anesthetic absorber during an anesthesia, which is carried out without the use of a volatile anesthetic, is not harmful. The wake-up criterion is modified in this embodiment such that the expiratory anesthetic concentration is not checked, but a time to wake up calculated from a three-compartment model for this intravenous agent is used (time to wake up), as this appears from DE 10 2004 050 717 B3.

Therefore, the respirating device may have, in each embodiment according to the present invention, a variant for operation with both a volatile anesthetic and propofol as an example of a non-volatile anesthetic. The basic embodiment of the device according to the present invention and its fundamental function are, however, identical to those described above.

The device according to the present invention may have, moreover, in another preferred embodiment, an electronically controlled evaporator for the anesthetic with an electronic mixer or a manual steamer with an electronic mixer or a purely manual dispensing system. When manually controlled components are used, the control signals of the wake-up algorithm are replaced by user instruction reports. These may optionally be acknowledged.

The device according to the present invention for respirating a patient may have a fan of a piston-and-cylinder unit or a system with turbine drive with or without circulation flow. Moreover, a conventional or a closed circulation system may be provided. Furthermore, the device may have a means for the manual or automated dispensing of the anesthetics.

The device according to the present invention may be designed for use with commercially available breathing tube accessories. The device is, furthermore, suitable for being operated with volatile anesthetic dispensing and/or intravenous anesthesia.

The object according to the present invention is also accomplished, furthermore, by the combination of the process as well as by the system. Since all the above-described advantages also apply in full measure to the process, reference is expressly being made here to the above discussion to avoid repetitions. Furthermore, it is pointed out that all the above-described embodiments can also be interpreted as variants of the processes. The description of these embodiments is therefore also disclosed, insofar as the person skilled in the art can identify them in the above discussion.

The present invention will be explained in more detail below on the basis of the drawings attached. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which the preferred embodiments of the invention are illustrated.

### BRIEF DESCRIPTION OF THE DRAWINGS

In the drawings:

FIG. 1 is a schematic view showing a simplified view of a device according to the present invention;

FIG. 2 is a flow chart showing a preparation algorithm for the wake-up process according to the invention;

FIG. 3 is an exemplary wake-up algorithm according to the invention; and

FIG. 4 is a perspective view showing an embodiment of a combination cartridge according to the present invention.

### DESCRIPTION OF THE PREFERRED EMBODIMENTS

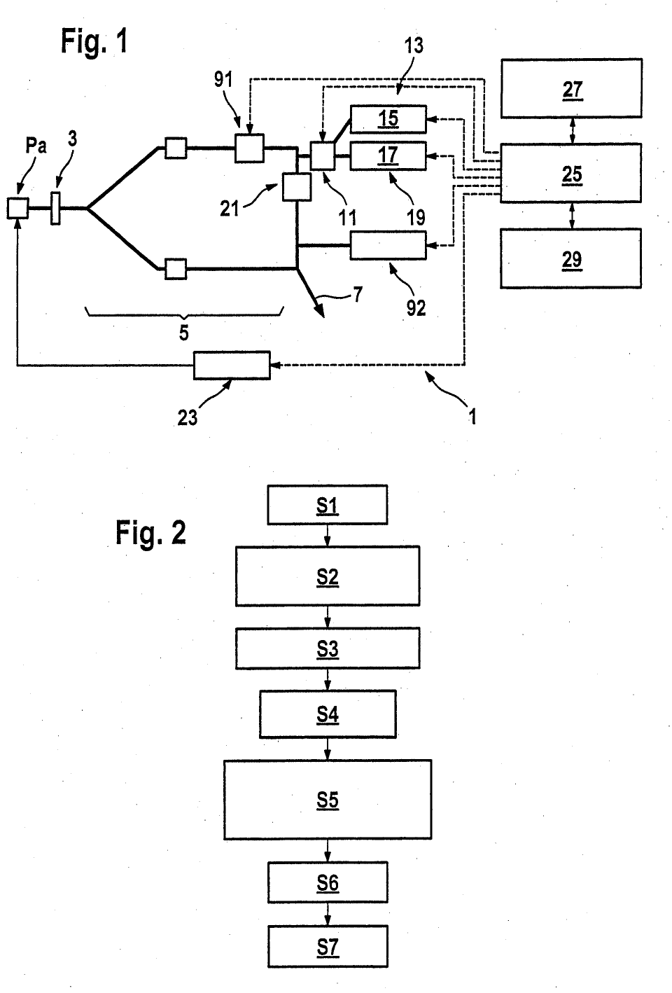

Referring to the drawings in particular, FIG. 1 schematically shows a simplified view of a device 1 according to the present invention for a patient Pa. The device 1 has a filter 3 with a rebreathing system 5 and with an outlet 7. Ventilation may take place in the device 1 by means of a fan 9, which may be arranged in FIG. 1 as an alternative as a closed circuit comprising a feed line and a drain line as a fan 91 or connected by a feed line as a fan 92. The fan variant 91 arranged in the inspiratory leg of the device 1 may be designed, for example, as a turbine or as a piston-and-cylinder unit. The fan variant 92 may likewise be provided as a piston-and-cylinder unit with a bellows drive. However, any other fan variant known to the person skilled in the art may likewise be used according to the present invention.

A combined fresh gas feed consisting of gas 15 and anesthetic 17 is designated by reference number 11. Gas is dispensed in the means 13.

Furthermore, FIG. 1 shows a means for anesthetic dispensing 19 with or without direct feed, as well as a filter element 21 for carbon dioxide and anesthetic, which filter may be provided in some of the embodiments according to the present invention. Additional dispensing or administration of an intravenously administered anesthetic may be carried out by means of a dispensing means 23.

It can be recognized from FIG. 1 that the operation of the device 1 can be carried out by means of a control unit 25 for the anesthesia fan 9. The control unit 25 may be connected to an operating and display unit 27. Reference number 29 designates a wake-up algorithm, which will be described below and acts on the control unit 25.

The filter 3 may be designed as a hygiene filter to avoid contamination of the device 1 by the respiration of a previous patient.

FIG. 2 shows a flow chart of a preparation algorithm for the wake-up process according to the present invention. The preparation algorithm comprises the steps S1 through S7, S1 signaling the start of the preparation algorithm. A current wake-up time or time to wake up as well as an optimal wake-up time are displayed in step S2. A suggestion for an optimal dosage of the intravenously administered as well as volatile anesthetics is calculated in step S3, after which the dosage is adapted in step S4. The adaptation may require acknowledgment by the user. After a waiting time, which ensures that the effect of the change is stable, the “prepared for wake-up process” report is sent in step S5. Optimization of the concentrations of the anesthetics used, as this is described in DE 10 2005 050 717 B3, is therefore concluded by this point in time. This optimization prevents, among other things, an overhang of opiates that were used. The current wake-up time is displayed in step S6, after which the preparation algorithm ends in step S7.

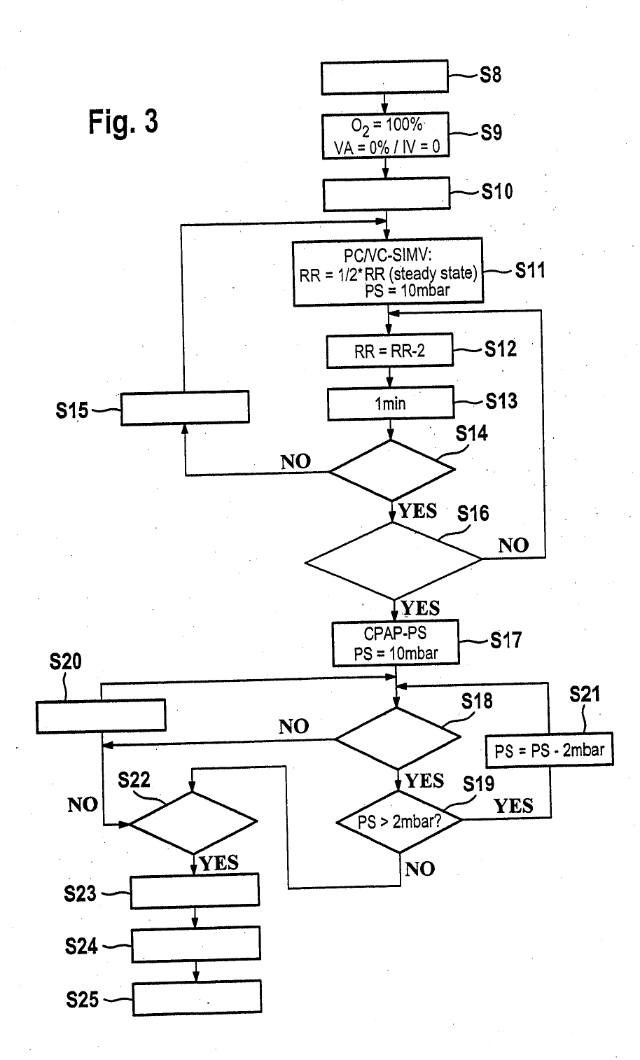

The wake-up process, which is shown in FIG. 3, begins with a step S8, which corresponds to the start time. In the next step S9, the oxygen concentration is raised to a sufficiently high value, whereas the concentrations of the volatile and intravenously administered anesthetics are reduced to zero. The breathing gas is passed through the absorber for anesthetics, whereas the CO₂ absorber is bypassed by means of the bypass in step S10. A pressure support is set in step 11 of FIG. 3 at 10 mbar for each spontaneous breath, and the respiration rate RR is set at half the respiration rate in “steady state.” The respiration rate is lowered by two units in step S12; this new state is maintained in step S13 without further changes, for example, over one minute before a set interruption criterion is checked in step S14. If the interruption criterion is not met (i.e., state “N”), a corresponding report is sent to the user in step S15, and the algorithm is set back to before step S11. However, if it is determined in step S14 that the interruption criterion is met (i.e., state “J”), the algorithm passes over to step S16, in which a checking is performed to determine whether the spontaneous minute volume—which can be measured, for example, from one breath to the next—is greater than 0.6 times the minute volume in the steady state or at equilibrium. If not, state “N” is set and the algorithm drops back to before step S12. However, if this criterion of step S16 is met and the spontaneous minute volume is above 0.6 times the minute volume in the steady state, this is considered to be state “J” and the algorithm passes over to step S17, in which a CPAP respiration takes place with a pressure support PS of 10 mbar. An observation is then performed in step S18 to determine whether the spontaneous minute volume (measured again, for example, from one breath to the next), increases. If yes (i.e., state “J”), the algorithm proceeds to step S20. If the algorithm has passed over to step S20, one waits for another breath before the algorithm returns to before step S18.

If, by contrast, the algorithm has passed over to step S19, a checking is performed to determine whether the pressure support PS is above 2 mbar. If yes, the algorithm (state “J”) proceeds to step S21, in which the pressure support PS is lowered by 2 mbar. However, if it is determined in step S19 that the pressure support is not greater than 2 mbar (state “N”), the algorithm passes over to step 22, in which the user is asked whether he or she would like to end the algorithm or the wake-up process. If the user answers this question with a “Yes” (state “J”), the bypass is switched back in step S23, the fresh gas flow for the spontaneous breathing is set to a high value in step S24, and the algorithm comes to an end in step S25. If, by contrast, a “No” answer (state “N”) is given in step S22, the algorithm passes again over to step S20.

It is noted that the processes shown in FIG. 3 are of a purely exemplary nature and that the values that were put out for the pressure support PS and other parameters and set values can be modified by the user of the device according to the present invention or the process according to the present invention and they shall not limit the present invention in any way.

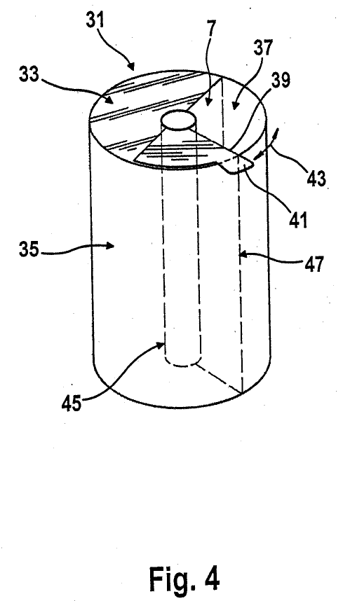

FIG. 4 shows a combination cartridge 31 (cartridge 31 for short) according to the present invention for use in treating the gas in the rebreathing system 5. In particular, the filter 21 can include the combination cartridge 31 shown in FIG. 4. In this embodiment, shown purely as an example in FIG. 4, the combination cartridge 31 has a fixed cover 33, which covers about ⅔ of the CO₂ absorber area of the cartridge in this embodiment. A section of the cartridge, which comprises about ¾ of the volume of the cartridge 31 and is filled with a CO₂ absorbent, is designated by the reference number 35. Reference number 37 designates the remaining ¼ of the volume of the cartridge 31, which is filled with activated carbon in the embodiment according to FIG. 4. The cartridge 31 has, furthermore, a mobile cover 39, by means of which the cartridge can be switched over from its action as a CO₂ absorber to the activated carbon operation for filtering the anesthetics, which flow through the cartridge 31 in the breathing gas.

A tongue designated by reference number 41 is used to turn the mobile cover 33 to switch over the cartridge 31 between its operating positions along the arrow designated by 43 in FIG. 4. As an alternative to the tongue adjustable manually, it is also possible to provide a changeover switch that can be actuated electrically. In the simplest case, the changeover switch is a stepping motor, which actuates the tongue.

The cartridge 31 has an inner tube 45, through which the breathing gas flows through the cartridge 31. Furthermore, the cartridge 31 has two partitions 47 for guiding the gas and storing the filter materials (i.e., the absorbent for CO₂ as well as for the anesthetics) separately.

Thus, the present invention proposes, for the first time ever, a device for shortening the wake-up phase of an anesthetized patient. Furthermore, a process for operating a device according to the present invention as well as a process for respirating a patient with gaseous anesthetic are proposed according to the present invention.

While specific embodiments of the invention have been shown and described in detail to illustrate the application of the principles of the invention, it will be understood that the invention may be embodied otherwise without departing from such principles.

What is claimed is:

1. A device for respirating a patient with a gaseous anesthetic, the device comprising:
a hypoventilation means for bringing about hypoventilation of the patient; and
a spontaneous breathing checking means for checking spontaneous breathing of the patient after hypoventilation has been brought about.

2. A device in accordance with claim 1, further comprising:
a carbon dioxide absorber; and
a switchable bypass position for said carbon dioxide absorber for bringing about hypoventilation of said patient in said bypass position.

3. A device in accordance with claim 2, further comprising another detector for detecting said bypass position.

4. A device in accordance with claim 1, further comprising a combination cartridge including a gaseous anesthetic absorber and a carbon dioxide absorber.

5. A device in accordance with claim 4, further comprising another detector for detecting an operational position of said combination cartridge.

6. A device in accordance with claim 1, further comprising preparation process means for carrying out a preparation process for preparing a wake-up phase of the patient from an anesthesia, wherein a composition of anesthetics administered can be optimized by said preparation process means.

7. A process for operating a device for respirating a patient with a gaseous anesthetic, the process comprising the steps of:
bringing about hypoventilation of a patient; and
checking spontaneous breathing of the patient when hypoventilation has been brought about.

8. A process in accordance with claim 7, wherein a carbon dioxide absorber is switched into a bypass position for bringing about hypoventilation of the patient.

9. A process in accordance with claim 8, wherein the composition of anesthetics is optimized in a preparation step for preparation of a wake-up process.

10. A respiration system, comprising:
a patient connection with an inspiration line and an expiration line;
a fresh gas feed including a breathing gas feed connected to said inspiration line;
a rebreathing system connected to said inspiration line and supplying a portion of breathing gas from said expiration line to said inspiration line;
a hypoventilation means for bringing about hypoventilation of a patient connected to said patient connection; and
a checking means for checking spontaneous breathing of the patient when hypoventilation has been brought about.

11. A system in accordance with claim 10, wherein said rebreathing system includes a carbon dioxide absorber with a bypass for fully or partially bypassing the carbon dioxide absorber in said rebreathing system and said hypoventilation means for bringing about hypoventilation of the patient changes a switching state of said bypass for fully or partially bypassing the carbon dioxide absorber.

12. A system in accordance with claim 11, wherein said carbon dioxide absorber in the rebreathing system includes a switchable bypass position for bringing about hypoventilation of the patient in said bypass position.

13. A system in accordance with claim 12, further comprising a detector for detecting said bypass position.

14. A system in accordance with claim 12, further comprising a combination cartridge including a gaseous anesthetic absorber and said carbon dioxide absorber, said combination cartridge being connected in said rebreathing system.

15. A system in accordance with claim 14, further comprising another detector for detecting an operational position of said combination cartridge.

16. A system in accordance with claim 11, further comprising an anesthesia administration means for administration of anesthesia to the patient.

17. A system in accordance with claim 16, further comprising a preparation process means for carrying out a preparation process for preparing a wake-up phase of the patient from anesthesia administration, wherein a composition of anesthetics administered can be optimized by said preparation process means.

18. A system in accordance with claim 16, wherein said anesthesia administration means feeds anesthesia to the patient via said fresh gas feed.
